(19) United States
(12) Patent Application Publication
(10) Pub. No.: US 2008/0009916 A1
(43) Pub. Date: Jan. 10, 2008

(54) APPLICATIONS OF HEART RATE VARIABILITY ANALYSIS IN ELECTROTHERAPY AFFECTING AUTONOMIC NERVOUS SYSTEM RESPONSE

(75) Inventors: Martin Rossing, Coon Rapids, MN (US); Robert S. Kieval, Medina, CA (US); Eric Erwin, Minneapolis, MN (US); Brad D. Pedersen, Minneapolis, MN (US); Vadim Braginisky, Eagan, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: CVRX, INC., Maple Grove, MN (US)

(21) Appl. No.: 11/750,936

(22) Filed: May 18, 2007

Related U.S. Application Data
(60) Provisional application No. 60/802,270, filed on May 19, 2006.

(51) Int. Cl. A61N 1/00

(52) U.S. Cl. 607/44

(57) ABSTRACT

A method of operating a baroreflex therapy system includes providing an implantable baroreflex activation device, providing a sensing arrangement, and providing a controller in operable communication with the baroreflex activation device and the sensing arrangement. The sensing arrangement is used to measure cardiac electrical activity of a patient to generate cardiac electrical activity data. The cardiac electrical activity data is communicated to the controller, wherein the controller performs heart rate variability analysis based on the cardiac electrical activity data. An indication of results of the heart rate variability analysis are provided, upon which a determination may be made to adjust a baroreflex therapy to be delivered by the implantable baroreflex activation device.

### CROSS-REFERENCE TO RELATED APPLICATIONS

This application claims the benefit of Provisional Patent Application No. 60/802,270, filed May 19, 2006, the disclosure of which is hereby incorporated by reference in its entirety.

### FIELD OF THE INVENTION

The invention relates generally to medical devices and methods, and more particularly, to baroreflex activation therapy devices, methods, and systems incorporating heart rate variability analysis for modulating a therapy.

### BACKGROUND OF THE INVENTION

Cardiovascular disease is a major contributor to patient illness and mortality. It also is a primary driver of health care expenditure, costing more than $326 billion each year in the United States. Hypertension, or high blood pressure, is a major cardiovascular disorder that is estimated to affect over 65 million people in the United Sates alone. Of those with hypertension, it is reported that fewer than 30% have their blood pressure under control. Hypertension is a leading cause of heart failure and stroke. It is the primary cause of death for tens of thousands of patients per year and is listed as a primary or contributing cause of death for hundreds of thousands of patients per year in the U.S. Accordingly, hypertension is a serious health problem demanding significant research and development for the treatment thereof.

Hypertension occurs when the body's smaller blood vessels (arterioles) constrict, causing an increase in blood pressure. Because the blood vessels constrict, the heart must work harder to maintain blood flow at the higher pressures. Although the body may tolerate short periods of increased blood pressure, sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke. Sustained high blood pressure may eventually result in an enlarged and damaged heart (hypertrophy), which may lead to heart failure.

Heart failure is the final common expression of a variety of cardiovascular disorders, including ischemic heart disease. It is characterized by an inability of the heart to pump enough blood to meet the body's needs and results in fatigue, reduced exercise capacity and poor survival. Heart failure results in the activation of a number of body systems to compensate for the heart's inability to pump sufficient blood. Many of these responses are mediated by an increase in the level of activation of the sympathetic nervous system, as well as by activation of multiple other neurohormonal responses. Generally speaking, this sympathetic nervous system activation signals the heart to increase heart rate and force of contraction to increase the cardiac output; it signals the kidneys to expand the blood volume by retaining sodium and water; and it signals the arterioles to constrict to elevate the blood pressure. The cardiac, renal and vascular responses increase the workload of the heart, further accelerating myocardial damage and exacerbating the heart failure state. Accordingly, it is desirable to reduce the level of sympathetic nervous system activation in order to stop or at least minimize this vicious cycle and thereby treat or manage the heart failure.

It has been known for decades that the wall of the carotid sinus, a structure at the bifurcation of the common carotid arteries, contains stretch receptors (baroreceptors) that are sensitive to the blood pressure. These receptors send signals via the carotid sinus nerve to the brain, which in turn regulates the cardiovascular system to maintain normal blood pressure (the baroreflex), in part through activation of the sympathetic nervous system. Electrical stimulation of the carotid sinus nerve has previously been proposed to reduce blood pressure and the workload of the heart in the treatment of high blood pressure and angina. For example, U.S. Pat. No. 6,073,048 to Kieval et al. discloses a baroreflex modulation system and method for stimulating the baroreflex based on various cardiovascular and pulmonary parameters. Implantable devices for treating high blood pressure or hypertension by stimulating various nerves and tissue in the body are known and described, for example, in U.S. Pat. No. 3,650,277 (stimulation of carotid sinus nerve), U.S. Pat. No. 5,707,400 (stimulation of vagal nerve), and U.S. Pat. No. 6,522,926 (stimulation of baroreceptors).

Implantable baroreflex activation devices and systems for treating hypertension generally include a pulse generator that stimulates the patient's baroreceptors by applying an electric field to the arterial wall of the carotid sinus artery via an electrode assembly intimately attached to the artery. The pulse generator is controlled by a microprocessor-based controller that may receive feedback from a sensed physiological parameter.

Conventionally, baroreflex activation therapy has been targeted to treating elevated blood pressure in the patient. To this end, known methods for obtaining the sensed physiological parameter have included measuring the arterial blood pressure with an implanted blood pressure sensing device, or monitoring blood pressure changes indirectly, such as by sensing cardiac rhythm information such as pulse rate.

Measurements of arterial blood pressure for controlling automatic baroreceptor activation devices can be difficult to obtain accurately by an implantable system having one or more sensors and measurement circuitry interfaced therewith. Although the cardiac rhythm is more readily measurable, such as with known electrocardiogram (ECG) measuring techniques, or with pulse timing, cardiac rhythm alone is only an indirect indicator of blood pressure. Additionally, an absolute measurement of a physiological parameter, that is, a measurement taken at a single moment in time, may be an incomplete indicator of the effectiveness of the therapy being delivered. Factors such as patient activity and/or environmental conditions may contribute to the sensed physiological parameter providing an incomplete representation of the condition of the patient and the effectiveness of the therapy.

One approach to monitoring heart rate during baroreflex therapy is to use a lead inserted into the heart of the patient. This approach can be overly complex, requiring additional invasive surgery to place the lead. Further, in many cases, the surgeon implanting the baroreflex therapy device is not trained to insert the lead in the heart, thereby requiring the services of an additional surgeon.

As various techniques are employed for treating cardiopulmonary and other diseases by electrical stimulation to influence the body's autonomic nervous system, there is an increasing need for assessing the effectiveness of, and controlling, the electrotherapy to produce certain desirable effects, and to minimize certain undesirable effects on the autonomic nervous system and on other bodily systems responsive to control via the autonomic nervous system.

### SUMMARY OF THE INVENTION

According to one aspect of the invention, heart rate variability (HRV) analysis is utilized to quantify the efficacy of baroreflex activation therapy (BAT) for the treatment of hypertension or other physiological condition or disease. In a related embodiment, the HRV analysis is further used to determine the need for increasing, decreasing, adjusting, stopping, or otherwise adjusting the BAT.

In various embodiments, the cardiac rhythm information collected for HRV analysis is gathered by a variety of sensing arrangements. For example, the cardiac rhythm can be sensed electrically such as according to known electrocardiogram (ECG) methods. In another example embodiment, the cardiac rhythm is detected by a pulse detection arrangement.

In a related embodiment, a combination of sensing arrangements of different types are used in concert, such as the combination of electrical cardiac rhythm sensing correlated to detected arterial pulses. This latter hybrid type of measuring arrangement can provide cardiac electrophysiology information in relation to heart contractility information, from which analytical inferences might be made about the patient's condition. For example, differences between the HRV as computed based on an ECG type measurement, versus the HRV as computed by a pulse detection arrangement may provide important diagnostic insight.

In a related embodiment, the BAT device is communicatively interfaced as part of a system capable of reporting physiologic conditions measured by the device to an output apparatus having a physician interface. This type of system arrangement can facilitate providing diagnostic information to the physician.

In one embodiment, the baroreflex activation device is configured with performance target information that is expressed in terms of HRV analysis output. If the actual measured patient condition (also in terms of HRV analysis output) is outside of a performance target range, the baroreflex stimulation device responds in some preconfigured manner. Responses include, for example, communicating the fact that the detected patient condition is out of a desirable range to an physician interface device; or adjusting the therapy in an attempt to improve the patient's condition through activation of the baroreflex system.

According to another aspect of the invention, HRV analysis is performed by a controller of a baroreflex activation device as part of a feedback system for controlling the administered therapy. In one type of embodiment, HRV-based feedback information can provide a more practicable ongoing measurement arrangement than a measurement of a sign (e.g. arterial blood pressure) that is the subject of treatment. In a related type of embodiment, HRV analysis is used in concert with other measured physiological responses or conditions. For example, HRV analysis can be considered by the BAT device or system together with one or more of: heart rate measurements, blood pressure measurements, respiration rate measurements, pulse oximetry measurements, blood chemistry measurements, ECG waveform analysis, end-tidal measurements, and the like.

In another type of embodiment, the autonomic nervous system response measurable by the HRV analysis is itself the subject of the treatment. In studying or treating diseases and conditions that have been correlated with certain autonomic nervous system conditions, or with one or more conditions expressed in terms of HRV analysis results, a BAT system can influence the body's physiology to achieve and maintain a certain state of the HRV as the target result.

One aspect of the invention recognizes that BAT effects the parasympathetic nervous system in addition to the sympathetic nervous system. Also, the baroreflex activation can affect the sympathetic/parasympathetic balance. According to one such embodiment, HRV analysis is performed in conjunction with BAT to measure effectiveness of the electrotherapy on different parts of the autonomic nervous system. For example, the HRV analysis can be utilized to distinguish the sympathetic response to the baroreflex activation from the parasympathetic response.

In a related embodiment, the BAT can be tuned or adjusted to emphasize a certain type of measured effect on the autonomic nervous system's response. For example, in one embodiment, the BAT can be controlled to produce a maximum suppressive effect on the sympathetic response, and a maximum enhancement effect on the parasympathetic response. In another embodiment, the electrotherapy can be adjusted to emphasize parasympathetic tone without regard to the effect on the sympathetic tone.

According to another aspect of the invention, BAT is applied to treating conditions that are affected by modulation of the body's sympathetic and parasympathetic activities. For example, according to various embodiments, the therapy is tuned or adjusted to slow or increase the heart rate, decrease or increase intestinal and gland activity, or relax or tighten sphincter muscles in the gastrointestinal tract. Examples of conditions that may be treatable by such modulation of the autonomic nervous system include, without limitation, sleep apnea, irritable bowel syndrome, Crohn's disease, incontinence, pain, and other disorders besides blood pressure-related conditions.

### BRIEF DESCRIPTION OF THE DRAWINGS

FIG. 1 is a diagram illustrating various components of an example baroreflex activation device that is implantable in a patient. according to one aspect of the invention.

FIG. 2 illustrates one embodiment of a central processing unit (CPU) of the baroreflex activation device of FIG. 1.

FIG. 3 illustrates an example control system according to one embodiment for regulating the autonomic nervous system based on patient monitoring and heart rate variability analysis.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Changes in blood pressure, heart rate, respiration, etc., are each an observable manifestation of the autonomic nervous system's response. Moreover, each named symptom is caused by a complex combination of physical responses. For example, blood pressure changes can be caused by arteriole constriction, cardiac rhythm activity, and numerous other mechanisms. As further research is conducted to study the root causes of hypertension and associated diseases, one type of treatment approach can be directed to targeting the greater autonomic nervous system response, rather than selected observable symptoms such as high blood pressure, heart rate, and the like.

Baroreflex activation therapy (BAT) is known to affect the body's autonomic nervous system, which in turn regulates the blood pressure, heart rate, respiration, and other systems. BAT may be used to activate baroreceptors to provide the brain with signals indicating an increase in blood pressure. These signals cause the brain to reduce the body's blood pressure and level of sympathetic nervous system and neurohormonal activation, and increase parasympathetic nervous system activation. The efficiency or effectiveness of BAT may be influenced by when it is delivered relative to the cardiac, respiratory and other respiratory cycles. For example, BAT may be more or less effective when delivered during the contraction vs. relaxation phase of the heart or during the expiratory vs. inspiratory phase of respiration. Accordingly, absolute measurements of arterial pressure or heart rate do not entirely reflect the full effects of baroreceptor stimulation.

The parasympathetic nervous system has a complementary relationship with the sympathetic nervous system. The body uses these two systems to regulate blood pressure. Stimulation or enhancement of the parasympathetic nervous system generally causes a decrease in blood pressure. Stimulating or enhancing the sympathetic nervous system, on the other hand, generally causes blood pressure to increase. If cardiac output is insufficient to meet demand (i.e., the heart is unable to pump sufficient blood), the brain activates a number of body systems, including the heart, kidneys, blood vessels, and other organs/tissues to correct this.

Baroreceptor signals in the arterial vasculature are used to activate a number of body systems which collectively may be referred to as the baroreflex system. For the purposes of the present invention, it will be assumed that the “receptors” in the venous and cardiopulmonary vasculature and heart chambers function analogously to the baroreceptors in the arterial vasculature, but such assumption is not intended to limit the present invention in any way. In particular, the methods described herein will function and achieve at least some of the stated therapeutic objectives regardless of the precise and actual mechanism responsible for the result. Moreover, the present invention may activate baroreceptors, mechanoreceptors, pressoreceptors, stretch receptors, chemoreceptors, or any other venous, heart, or cardiopulmonary receptors which affect the blood pressure, nervous system activity, and neurohormonal activity in a manner analogous to baroreceptors in the arterial vasculation. For convenience, all such venous receptors will be referred to collectively herein as “baroreceptors” or “receptors” unless otherwise expressly noted.

While there may be small structural or anatomical differences among various receptors in the vasculature, for the purposes of some embodiments of the present invention, activation may be directed at any of these receptors and/or nerves and/or nerve endings from these receptors so long as they provide the desired effects. In particular, such receptors will provide afferent signals, i.e., signals to the brain, which provide the blood pressure and/or volume information to the brain. This allows the brain to cause “reflex” changes in the autonomic nervous system, which in turn modulate organ activity to maintain desired hemodynamics and organ perfusion. Stimulation of the baroreflex system may be accomplished by stimulating such receptors, nerves, nerve fibers, or nerve endings, or any combination thereof.

Techniques are known for measuring the sympathetic and parasympathetic nervous system responses. Beat-to-beat fluctuations which occur around a person's mean heart rate are known as heart rate variability (HRV). The fluctuations from beat-to-beat are attributed, in part, to the nonlinear interaction between the sympathetic and parasympathetic branches of the autonomic nervous system. The sympathetic autonomic and parasympathetic autonomic nervous systems regulate, to some extent, the sinoatrial (SA) node and atrioventricular (AV) node of the heart and, thus, largely influence the control of the heart rate. These two nervous systems operate somewhat reciprocally to effect changes in the heart rate. In this regard, parasympathetic stimulation decreases the firing rate of the pacing cells located in the sinus node of the heart. Sympathetic stimulation, on the other hand, increases this firing rate.

Many clinicians agree that the parasympathetic and sympathetic inputs to the SA node mediate low frequency heart rate fluctuations (i.e., generally below 0.15 Hz), whereas modulation of parasympathetic outflow mediates higher frequency fluctuations. Studies have further shown that a decrease in heart rate variability correlates with a decrease in parasympathetic nervous activity and an accompanied increase in sympathetic nervous activity. See J. Thomas Bigger, et al, “Components of Heart Rate Variability Measured During Healing of Acute Myocardial Infarction” American Journal of Cardiology, Vol. 61 (1988), pp. 208-215. In a healthy, resting heart, for instance, the parasympathetic activity dominates to maintain the heart rate. However, in an unhealthy heart, for example one having heart disease, sympathetic activity may more influence and control the heart rate.

HRV analysis can be performed in at least two ways, such as by using time domain or frequency domain measures of variability. Commonly used time domain measures of HRV are concerned with the variability of the interval between the R waves for heart beats with a normal sinus mechanism (NN intervals). Two commonly used measures are the standard deviation of NN intervals (SD), which increases with a reduction in sympathetic tone; and the root mean square of successive differences between adjacent NN intervals (rMSSD), which increases as parasympathetic tone is enhanced.

Frequency domain measures of heart rate variability are typically obtained by performing Fourier analysis, such as fast Fourier transformation (FFT) on sampled sets of ECG recordings, and analyzing changes in the content of certain frequency bins as a function of time. Two peaks are typically present in the FFT of five-minute ECG recordings, although ECG recordings may be taken over a longer or shorter period of time as deemed necessary. High frequency (HF) (0.15-0.40 Hz) peaks reflect modulation of the efferent parasympathetic activity, and low frequency (0.04-0.15 Hz) (LF) peaks reflect modulation of the efferent parasympathetic vagal and efferent sympathetic nervous system. The amplitude of LF or HF power is a measure of autonomic nervous system modulation of sinus node firing, and not a measure of global sympathetic and parasympathetic nervous system tone; however, the LF/HF ratio is used as an index of sympathetic parasympathetic balance. In normal subjects the amplitude of LF power exceeds that of HF; however, during controlled respiration there is a marked increase in HF and a reduction in the LF components and of the LF/HF ratio.

Following acute beta-adrenergic blockade with the nonselective betablocker propranolol, which would be expected to result in peripheral sympathoinhibition, there is typically an increase in the HF component and a reduction in the LF component of the FFT of five-minute ECG recordings. This is associated with a reduction in the LF/HF ratio. When blood pressure is reduced by an intravenous infusion of nitroglycerine, or tilt testing, there is typically an increase in the LF component indicating sympathetic activation.

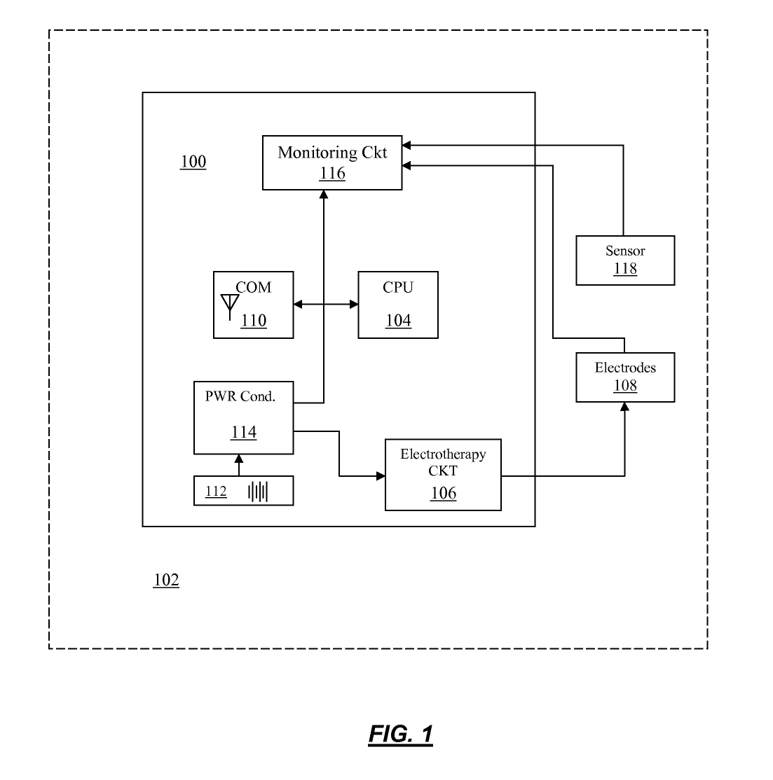

FIG. 1 is a diagram illustrating an example baroreflex activation device 100 that is optionally implantable in a patient 102. Persons of ordinary skill in the art will recognize that the aspects of the invention can be suitably applied to non-implantable, i.e. external baroreflex activation devices. Device 100 includes a central processor unit (CPU) 104, which may include one or more microprocessors or microcontrollers, for example, that is configured to control the operation of the device. CPU 104 is configured to cause the device to administer the therapy via electrotherapy circuit 106 and electrodes 108. A communications circuit 110 is interfaced with CPU 104 and is used for communicating information between CPU 104 and equipment external to the patient 102, such as a device programmer (not shown), external processor, or external or remote sensors (not shown). Baroreflex activation device 100 also includes a power source such as a battery 112, and power conditioning circuitry 114 for converting the battery power into various power supplies suitable for powering each component or sub-system of the device. CPU 104 can detect at least one physiologic condition of patient 102 via patient monitoring circuitry 116 and at least one sensor 118. In one embodiment, CPU 104 detects at least one physiologic parameter indicative of the heart rate of patient 102 via patient monitoring circuitry 116 and at least one sensor 118.

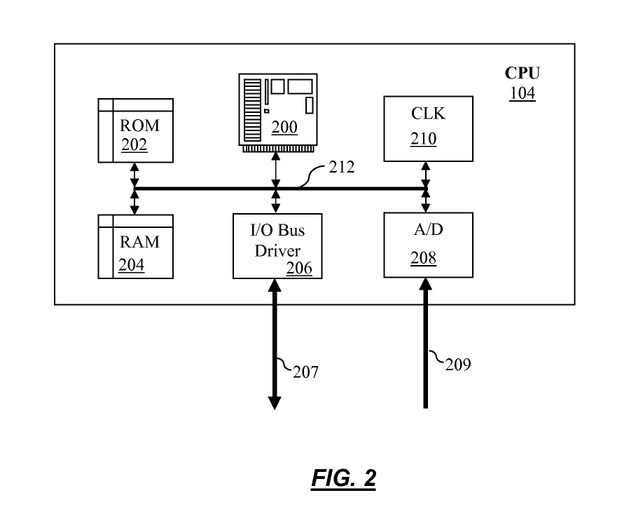

FIG. 2 illustrates one embodiment of CPU 104. CPU 104 includes a microprocessor core 200; read-only memory (ROM) 202 for storing instructions; random access memory (RAM) 204 for use as data gathering, or scratchpad memory during operation; input/output (I/O) bus driving circuitry 206 for transmitting and receiving information via, and controlling the use of, I/O bus 207; analog-to-digital (A/D) converter 208 for converting analog signals received via analog inputs 209 into a digital format for use by microprocessor core 200; and clock 210 for providing a time base for use by microprocessor core 200. In one type of embodiment, CPU 104 has signal processing capability (such as that provided by a DSP core) to perform computations on relatively long sequences of sampled data. An internal CPU interconnect 212 provides an interface between the various CPU components, and can include conventional data exchange hardware, such as a data bus, an address bus, and control lines (not shown).

Referring again to FIG. 1, in a related embodiment, the patient monitoring circuitry 116, or at least a portion of the signal processing circuitry of CPU 104 is situated remotely from device 100 and communicatively coupled with device 100. Similarly, sensor 118 can be remotely situated from patient monitoring circuitry 116 or from device 100. In a further related embodiment, electrodes 108 may be remotely situated from device 100.

Sensor 118 can take many forms within the spirit of the invention. For example, sensor 118 can include an intravascular or external pressure transducer, arterial pulse detector ultrasonic activity detector, or any suitable device, internal or external to the patient, for sensing or detecting mechanical events or activity of the patient. In other embodiment sensor 118 can be a chemical or optical sensor, such as a sensor for measuring a degree of blood oxygenation. Sensor 118 may also comprise a cardiac electrical activity detector, such as one or a set of ECG probes, whether internal or external to the patient. The ECG probes can be of the near-field type that are situated proximally (within 1-2 cm) of the heart or inside the heart. The ECG probes can also be of the far-field or extracardial type, such as external patches or subcutaneously-implanted electrodes. Sensor 118 can also comprise a set of individual sensors of the same type or of different types. Sensor 118 may be implanted in whole or in part, or may be disposed outside the body.

According to one embodiment of the invention, patient monitoring circuitry 116 operates in cooperation with sensor 118 to collect cardiac activity information for CPU 104. CPU 104 processes this cardiac activity information to produce a characterization of the patient's condition being monitored. In one embodiment, monitoring circuitry 116 and sensor 118 collect cardiac rhythm information, such as the time difference between R-wave peaks, or the period or frequency of detected arterial pulses or heart beats. Such data may be communicated from sensor 118 to patient monitoring circuitry and/or CPU 104 in a variety of ways, including direct communication through cables and wires, via wireless communication methods, such as radio frequency, infrared, and other technologies, or through other techniques known in the art, and depending upon type and location of sensor 118. CPU 104 analyzes this cardiac rhythm information according to heart rate variability (HRV) analysis techniques, such as those described above, and produces an evaluated score or some other quantitative assessment of the HRV analysis.

In one embodiment, a combination of electrical cardiac rhythm sensing is correlated to detected arterial pulses. This type of scheme can provide cardiac electrophysiology information in relation to heart contractility information. Processor 104 can use this information to make additional inferences or diagnoses of the patient's condition. For example, differences between the HRV as computed based on by an ECG type measurement, versus the HRV as computed by a pulse detection arrangement may provide important diagnostic insight in to a systemic cause of an observable disease.

In a related embodiment, processor 104 conducts HRV analysis so as to distinguish the effectiveness of the BAT as affecting the sympathetic nervous system response, or as affecting the parasympathetic nervous system response. This degree of analytical insight can be instituted in concert with other, symptomatic-oriented, physiological sensing such as blood pressure, pulse oximetry, and the like. Processor 104 can further process these various physiological measurements or characterizations to synthesize the different types of information into a comprehensive patient condition assessment. Analytical methods can include regression analysis, morphology, and other computational techniques that are known in the art.

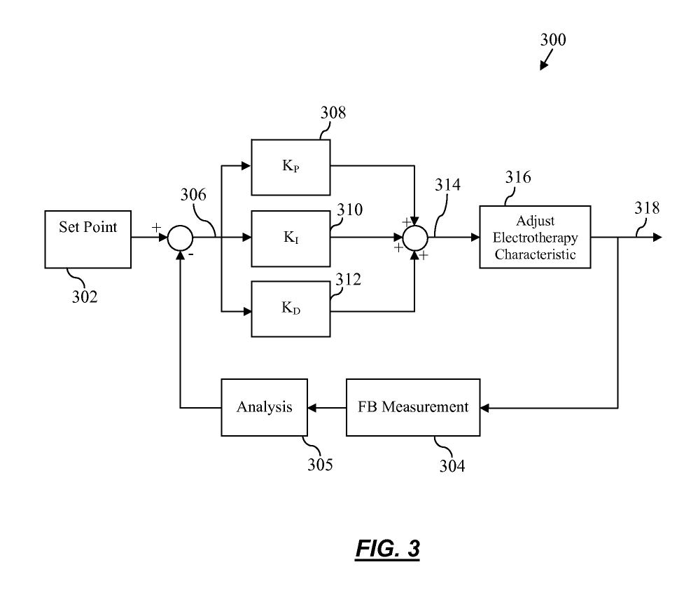

In one embodiment, as described above, baroreflex activation device 100 operates a closed-loop control system for adjusting one or more electrotherapy characteristics to achieve a desired result as measured by patient monitoring circuitry 116 and sensor 1 18. FIG. 3 illustrates an example control system 300 for regulating the therapy characteristics to produce a desired effect on a monitored physiological parameter. A set point 302 representing the desired physiological condition is provided to the system as depicted. Set point 302 is a target that system 300 will strive to achieve by adjusting the level, waveform, or any other characteristic or combination of characteristics of the electrotherapy administration. The adjustment can be conducted according to a predetermined regime or algorithm. Set point 302 can be stable, or time-variable, depending on the nature of the physiologic condition to be controlled. For additional disclosure pertaining to therapy characteristics that can be adjusted by system 300 to achieve set point target 302, see U.S. Pat. No. 6,985,774 to Kieval et al., the disclosure of which is hereby incorporated by reference in its entirety.

Control system 300 compares set point 302 with an actual measurement 304 and analytical assessment 305 of the measurement 304 to produce an error signal 306. The error signal 306 is operated on by proportional-integral-differential controls 308, 310, and 312, respectively. Proportional control 308 includes a proportional weighting constant KP; integral control 310 includes an integral weighting constant K1; and differential control 312 includes differential weighting constant KD. The output of each control type is aggregated to produce a control signal 314. The baroreflex activation device administers a therapy dosage 316 according to the control signal 314, which results in a controlled effect 318 in the patient.

In one example embodiment, the feedback loop includes the analytical result of the HRV analysis. Thus, for example, in an embodiment where the control objective is to operate the baroreflex stimulation signal in a mode that produces the greatest parasympathetic tone enhancement in the patient, a measured and computed rMSSD is produced by analytical assessment 305 and used as the feedback signal to compare against a desired set point 302 expressed in terms of rMSSD.

Various techniques for BAT can be applied according to embodiments of the invention to achieve different types of effects on the patient's autonomic nervous system. For example, in addition to, or in place of stimulating receptors in the carotid sinus artery at the carotid bifurcation, baroreflex therapy can be applied to the carotid body to stimulate other receptors. Stimulation of the carotid body can produce an effect on the autonomic nervous system that generally opposes the effects resulting from stimulation of the baroreceptors.

In another type of embodiment, the autonomic nervous system response measurable by the HRV analysis is itself the subject of the treatment. Thus, according to one aspect of the invention, autonomic nervous system condition or response is used as part of a control loop capable of (a) suppressing sympathetic tone and enhancing parasympathetic tone; (b) enhancing sympathetic tone and suppressing parasympathetic tone; (c) enhancing or suppressing sympathetic tone; or (d) enhancing or suppressing parasympathetic tone. Embodiments of this aspect include treatment of patients with sleep disorders such as sleep apnea, in which it may be desirable to increase the patient's sympathetico-adrenal response during the day, or during active times, and to decrease it during times of rest.

To selectively enhance or suppress sympathetic vs. parasympathetic response, an implanted baroreflex activation device with multiple electrode assemblies can be utilized, with the first electrode assembly positioned to stimulate baroreceptors at a first location, the carotid bifurcation, and the second electrode assembly positioned to stimulate baroreceptors at a second location. In a related embodiment, a single electrode assembly with a plurality of electrode sets includes a first electrode set positioned to stimulate baroreceptors in a first area or location, while the second electrode set is positioned to stimulate baroreceptors in a second area or location.

In another embodiment, in which the activity of the digestive system is influenced, for example, the baroreflex activation device selectively stimulates one or the other of these sensory mechanisms to produce the desired effect. Thus, to increase digestive activity, baroreflex activation can be administered to enhance the parasympathetic tone and suppress the sympathetic tone; and to decrease digestive activity, the carotid body can be stimulated to suppress the parasympathetic tone while enhancing the sympathetic tone.

In another embodiment, multiple baroreflex activation devices can be provided as part of a baroreflex activation therapy system. In one embodiment, the sensing arrangement may comprise a separate baroreflex activation device that is capable of delivering cardiac rhythm management (CRM). Additional disclosure pertaining to the combination of BAT devices and therapies with CRM devices and therapies that is relevant to the present invention can be found in Published U.S. Patent Application No. 2006/0004417 to Rossing et al., and Published U.S. Patent Application No. 2006/0074453 to Kieval et al., the disclosures of which are hereby incorporated by reference in their entirety.

Additional disclosure material that exemplifies at least a portion of the other features and functionality of the range of embodiments within the spirit and scope of the present invention can be found in Published U.S. Patent Application No. 2005/0154418 to Kieval et al., Published U.S. Patent Application No. 2005/0251212 to Kieval et al., and Published U.S. Patent Application No. 2006/0293712 to Kieval et al., the disclosures of which are hereby incorporated by reference in their entireties. Additional disclosure material relating to vascular anatomy and the cardiovascular system as it pertains to the present invention can be found in U.S. Pat. No. 6,522,926 to Kieval et al., the disclosure of which is hereby incorporated by reference.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A method of operating a baroreflex therapy system, comprising:
providing an implantable baroreflex activation device;
providing a sensing arrangement;
providing a controller in operable communication with the baroreflex activation device and the sensing arrangement;
measuring cardiac electrical activity of a patient with the sensing arrangement to generate cardiac electrical activity data;
communicating the cardiac electrical activity data to the controller;
performing heart rate variability analysis with the controller based on the cardiac electrical activity data; and
providing an indication of results of the heart rate variability analysis upon which a determination may be made to adjust a baroreflex therapy to be delivered by the implantable baroreflex activation device.

2. The method of claim 1, wherein providing an indication of the results of the heart rate variability analysis is selected from the group consisting of: communicating the results to a physician interface device, and communicating the indication to the implantable baroreflex activation device.

3. The method of claim 1, wherein the controller is configured with a desired performance target range, further comprising:
comparing the results of the heart rate variability analysis to the desired performance target range; and
automatically adjusting the baroreflex therapy with the controller if the results of the heart rate variability analysis are outside of the desired performance range.

4. The method of claim 1, wherein the target performance range is set by a physician.

5. The method of claim 1, further comprising:
sensing a physiological parameter associated with heart rate to generate physiological data;
communicating the physiological data to the controller; and
providing an indication of the physiological data and results of the heart rate variability analysis, upon which a determination may be made to adjust the baroreflex therapy to be delivered by the baroreflex activation device.

6. A system for providing a baroreflex therapy, comprising:
a baroreflex activation device;
a sensing arrangement adapted to collect cardiac electrical activity data; and
a controller in communication with the device and the sensing arrangement, wherein the controller is adapted to receive the cardiac electrical activity data from the sensing arrangement, perform heart rate variability analysis of the cardiac electrical activity data, and provide an indication of results of the heart rate variability analysis upon which a determination may be made to adjust a baroreflex therapy to be delivered by the baroreflex activation device.

7. The system of claim 6, further comprising a physician interface device communicatively coupled to the controller and configured to communicate the results of the heart rate variability analysis to a physician.

8. The system of claim 6, wherein the controller includes a desired performance target range, the results of the heart rate variability analysis are compared to the desired performance target range, and the controller is adapted to automatically adjust the baroreflex therapy if the results of the heart rate variability analysis are outside of the desired performance target range.

9. The system of claim 6, wherein the sensing arrangement includes an additional sensor adapted to sense a physiological parameter associated with heart rate to generate physiological data, the physiological data communicated to the controller for providing an indication of the physiological data and results of the heart rate variability analysis, upon which a determination may be made to adjust the baroreflex therapy to be delivered by the baroreflex activation device.

10. A system for providing a baroreflex therapy, comprising:
a baroreflex activation device;
means for sensing cardiac electrical activity of a patient, adapted to collect an electrocardiogram; and
means for controlling the system in communication with the device and the means for sensing cardiac electrical activity, wherein the means for controlling is adapted to receive the cardiac electrical activity data from the means for sensing, perform a heart rate variability analysis of the cardiac electrical activity data, and provide an indication of results of the heart rate variability analysis upon which a determination may be made to adjust a baroreflex therapy to be delivered by the baroreflex activation device.

11. The system of claim 10, further comprising a means for communicating the results of the heart rate variability analysis to a physician.

12. The system of claim 10, wherein the means for controlling includes a desired performance target range, the results of the heart rate variability analysis are compared to the desired performance target range, and the means for controlling is adapted to automatically adjust the baroreflex therapy if the results of the heart rate variability analysis are outside of the desired performance target range.

13. The system of claim 10, wherein the means for sensing is adapted to sense a physiological parameter associated with heart rate to generate physiological data, wherein the physiological data is communicated to the controller to provide an indication of the physiological data and results of the heart rate variability analysis upon which a determination may be made to adjust the baroreflex therapy to be delivered by the baroreflex activation device.

14. A method, comprising:
providing an implantable baroreflex activation device;
providing a sensing arrangement;
providing a controller operably communicable with the baroreflex activation device and the sensing arrangement; and
providing instructions for operating the device, comprising:
measuring cardiac electrical activity of a patient with the sensing arrangement to generate cardiac electrical activity data;
communicating the cardiac electrical activity data to the controller;
performing heart rate variability analysis with the controller based on the cardiac electrical activity data; and
providing an indication of the results of the heart rate variability analysis to determine an effect on the autonomic nervous system of a patient, upon which a determination may be made to adjust a baroreflex therapy to be delivered by the implantable baroreflex activation device.

15. The method of claim 14, wherein the heart rate variability analysis is used to distinguish a sympathetic nervous system response to the baroreflex therapy from a parasympathetic nervous system response to the baroreflex therapy.

16. The method of claim 14, wherein the baroreflex therapy is adjusted in response to the results of the heart rate variability analysis to increase and/or suppress an effect on the autonomic nervous system.

17. The method of claim 14, wherein the controller is configured with a desired performance target range of an autonomic nervous system response, further comprising:
comparing the results of the heart rate variability analysis to the desired performance target range; and
automatically adjusting the baroreflex therapy with the controller if the results of the heart rate variability analysis are outside of the desired performance range.

18. The method of claim 14, wherein the baroreflex therapy is adjusted in response to the results of the heart rate variability analysis to increase an enhancement effect on the parasympathetic nervous system of the patient.

19. The method of claim 14, wherein the baroreflex therapy is adjusted in response to the results of the heart rate variability analysis to increase a suppressive effect on the sympathetic nervous system of the patient.

20. The method of claim 14, wherein providing an indication of the results of the heart rate variability analysis is selected from the group consisting of: communicating the results to a physician interface device, and communicating the results to the implantable baroreflex activation device.

21. A baroreflex therapy system, comprising:
an implantable baroreflex activation device;
a sensing arrangement;
a controller in operable communication with the baroreflex activation device and the sensing arrangement; and
instructions recorded on a tangible medium for operating the device, comprising:
measuring cardiac electrical activity of a patient with the sensing arrangement to generate cardiac electrical activity data;
communicating the cardiac electrical activity data to the controller;
performing heart rate variability analysis with the controller based on the cardiac electrical activity data; and
providing an indication of the heart rate variability analysis upon which a determination may be made to adjust a baroreflex therapy to be delivered by the implantable baroreflex activation device.

22. The system of claim 21, wherein providing an indication of the results of the heart rate variability analysis is selected from the group consisting of: communicating the results to a physician interface device, and communicating the results to the implantable baroreflex activation device.

23. The system of claim 21, wherein the controller is configured with a desired performance target range, further comprising:
comparing the results of the heart rate variability analysis to the desired performance target range; and
automatically adjusting the baroreflex therapy with the controller if the results of the heart rate variability analysis are outside of the desired performance range.

24. The system of claim 21, further comprising:
sensing a physiological parameter in addition to the cardiac electrical activity;
combining the physiological parameter and the heart rate variability analysis upon which a determination may be made to adjust the therapy.

25. The system of claim 21, wherein the sensing arrangement is selected from the group consisting of:
at least one sensor integrated with the implantable baroreflex activation device,
at least one sensor separate from the implantable baroreflex activation device,
a second implantable medical device that is capable of delivering cardiac rhythm management, and
a sensor integrated with the baroreflex activation device, the device also capable of delivering cardiac rhythm management.
